(19) United States
(12) Patent Application Publication
(10) Pub. No.: US 2019/0201547 A1
(43) Pub. Date: Jul. 4, 2019

(54) Nanoparticle-Based Method for Real-time Actuated Release and Monitoring of Cargos to Cells

(71) Applicant: The Government of the United States of America, as represented by the Secretary of the Navy, Arlington, VA (US)

(72) Inventors: James B. Delehanty, Washington, DC (US); Lauren D. Field, College Park, MD (US); Igor L. Medintz, Springfield, VA (US); Scott Walper, Springfield, VA (US); Kimihiro Susumu, Alexandria, VA (US); Guillermo Lasarte-Aragones, Washington, DC (US)

(73) Assignee: The Government of the United States of America, as represented by the Secretary of the Navy, Arlington, VA (US)

(21) Appl. No.: 16/236,899

(22) Filed: Dec. 31, 2018

Related U.S. Application Data
(60) Provisional application No. 62/612,912, filed on Jan. 2, 2018.

(51) Int. Cl. A61K 47/69; A61K 31/704; A61K 49/00; A61K 47/65

(52) U.S. Cl. CPC A61K 47/6951; A61K 47/6929; A61K 47/65; A61K 49/0093; A61K 31/704

(57) ABSTRACT

A new nanoparticle (NP)-based, multicomponent delivery/reporter construct can mediate the controlled, spatiotemporal, active release of an appended cargo to the cytosol of mammalian cells. The construct comprises components including (1) a central NP scaffold, for example a photoluminescent quantum dot (QD); (2) a bridging structure that self-assembles to the NP surface (for example, histidine-tagged maltose binding protein); and (3) a cargo, for example a ligand-dye/drug conjugate, incorporating a ligand that allows the cargo to releasably bind to the bridging structure (e.g., a β-cyclodextrin ligand for binding to maltose binding protein).

### CROSS-REFERENCE TO RELATED APPLICATIONS

This application claims the benefit of U.S. Provisional Patent Application No. 62/612,912 filed on Jan. 2, 2018, the entirety of which is incorporated herein by reference.

### BACKGROUND

The controlled, targeted in vivo delivery of cargos such as therapeutics and imaging agents continues to be an intensely active area of research. A primary goal is to increase the efficacy or therapeutic index of the delivered therapeutic; i.e., increase the specific, localized activity of the therapeutic while decreasing its off-target toxicity. In this context, the unique size-dependent attributes of nanoparticle (NP) materials make them attractive as delivery vehicles given their (1) small size, (2) tissue penetration, (3) circulation time and clearance, and (4) ability to have the release of their on-board cargo controlled by any of a number of “actuation” modalities.

NP actuation modalities broadly fall into two classes, based on the mechanism of cargo release: passive and active. Passive modalities typically rely on the efflux of cargo from the NP surface or core and lack the ability to tightly control when and where the cargo is released. Active modalities rely on an external stimulus (for example, light, magnetic field, radio waves, or innate cellular physiology such as pH or enzymatic activity) to cause cargo release. Currently, one the main limitations of active modalities is the lack of spatiotemporal control over the triggered release of the NP cargo. Visible and near infrared light, common triggers in active NP actuation, have limited tissue depth penetration. Hence, a need exists for new NP active actuation modalities that offer enhanced spatial and temporal control over triggered cargo release coupled with little to no associated toxicity of the NP construct prior to triggered cargo release.

### BRIEF SUMMARY

In a first embodiment, a nanoparticle construct includes a nanoparticle configured as a central scaffold; a molecule configured as a bridging structure and comprising a first binding element that allows attachment to the nanoparticle and a second binding element distinct from the first binding element; and a cargo incorporating a ligand configured to allow for releasable binding of the cargo from the second binding element of the bridging structure.

In a further embodiment, a nanoparticle construct includes a quantum dot configured as a central scaffold; maltose binding protein configured as a bridging structure and comprising a domain for binding to the quantum dot; and a cargo incorporating a β-cyclodextrin ligand configured to allow for releasable binding of the cargo from the maltose binding protein.

In another embodiment, a method of delivery includes providing a construct as described herein and contacting the construct with an analog of the ligand, thereby resulting in release of the cargo from the construct.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A through 1D illustrate an exemplary quantum dot (QD) protein-ligand system for active cargo actuation. FIG. 1A is a schematic of the QD-maltose binding protein β-cyclodextrin (BCD)-TideFluor3 (TF3) nanoconjugate for active, spatiotemporal actuation of intracellular cargo release. The assembled construct is designed to facilitate FRET between the QD donor and the BCD-TF3 acceptor. After addition of the competitive inhibitor maltose, the TF3 is released from the binding pocket. This is coupled with increasing QD donor PL and decreased PL from the dye acceptor. FIG. 1B is a schematic of another active delivery system analogous to that shown in FIG. 1A where the BCD-DOX is used as the ligand in the MBP binding pocket. DOX is an efficient but poorly emissive acceptor for the QD; in the intact complex it quenches QD PL with negligible PL from the DOX. FIG. 1C shows the spectra of QD and TF3 absorbance and emission showing the overlap of the donor emission and the acceptor absorbance for this FRET pair. FIG. 1D is a schematic of the β-cyclodextrin-DOX synthesized for the actuation modality.

FIGS. 2A through 2D show the use of FRET to confirm the active actuation of QD-protein-ligand systems. FIG. 2A shows a spectral analysis of QD-MBP-BCD-TF3 conjugate in multiple wells of a microtiter plate show efficient quenching of the QD donor and sensitized emission of the TF3 acceptor. B) Addition of increasing concentrations of maltose results in decrease of FRET and increased QD re-emission coupled with a decrease in TF3 sensitized emission. C) Similar spectral analysis of QD-MBP-BCD-DOX shows quenching of QD PL. No emission is seen for the poorly emissive DOX. D) Addition of maltose results in QD re-emission in a dose-dependent manner (DOX is minimally emissive in this system

FIGS. 3A and 3B provide a demonstration of active actuation of QD-MBP-BCD-TF3 construct in live cells. FIG. 3A shows spectral images of COS-1 cells microinjected with the QD-MBP-BCD-TF3 construct. Shown are time-resolved spectral images after addition of 25 mM maltose to the cellular medium. Note the progression of yellow emission (TF3) at t₀ min to green emission (QD) at t₃₀ min. FIG. 3B shows spectra derived from time-resolved imaging after addition of maltose to the cellular medium. Each trace corresponds to the “minutes post-maltose” addition as indicated. Similar experiments were performed for the BCD-DOX ligand system (vide infra).

FIGS. 4A and 4B show the quantitative analysis of intracellular maltose-induced ligand release. FIG. 4A provides time-resolved spectral images of QD-MBP-BCD-TF3 response to increasing concentrations of extracellular maltose. Plots show the ratios of donor/acceptor (D/A) emission versus maltose concentration as a function of time (in minutes). Increasing D/A indicates increased displacement of the BCD-TF3 ligand from the MBP binding pocket. FIG. 4B is a time-resolved plot of QD donor re-emission (normalized) for the QD-MBP-BCD-DOX assembly as a function versus maltose concentration as a function of time. Donor re-emission was determined using the identity, 1−(F_{DA}/F_{D}), where F_{DA} is the PL of the QD donor in the presence of the DOX quencher and F_{D} is the QD PL in the absence of the DOX quencher. Increasing QD donor re-emission indicates maltose-induced release of the BCD-DOX from the MBP protein.

### DETAILED DESCRIPTION

### Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

As used herein, the term “nanoparticle” refers to a particle having a largest dimension of no greater than 100 nanometers

### Overview

A new nanoparticle (NP)-based, multicomponent delivery/reporter construct can mediate the controlled, spatiotemporal, active release of an appended cargo to the cytosol of mammalian cells. The construct comprises components including (1) a central NP scaffold, for example a photoluminescent quantum dot (QD); (2) a bridging structure that self-assembles to the NP surface (for example, histidine-tagged maltose binding protein); and (3) a cargo, for example a ligand-dye/drug conjugate, incorporating a ligand that allows the cargo to releasably bind to the bridging structure (e.g., a β-cyclodextrin ligand for binding to maltose binding protein).

In one embodiment, the central NP scaffold and the dye/drug portion of the ligand-dye/drug conjugate engage in Förster resonance energy transfer (FRET) wherein the photoexcited NP donor transfers energy to the proximal dye/drug. After the construct is introduced into the cellular cytosol, the active release or “actuation” of the ligand-dye/drug cargo is induced by the simple addition of a “competitive structural analog” to the extracellular medium. The competitive analog is internalized into cells by natural cellular uptake processes and drives the concentration-dependent release of the ligand-dye/drug conjugate from the protein binding pocket. FRET can be used to monitor and optically report on the intracellular release of the ligand in real-time by tracking the time- and maltose concentration-dependent changes in donor and acceptor photoluminescence (PL).

### Examples

Two example platforms are described below. In both cases, the ligand is the cycloamylose, β-cyclodextrin, conjugated to a cargo which is either a highly emissive dye acceptor (TideFluor 3) or the minimally emissive anti-cancer drug, doxorubicin. The β-cyclodextrin releasably binds to maltose binding protein (MBP) modified with a polyhistidine tail (a first binding element), binding at the binding pocket (the second binding element) of the MBP. The competitive structural analog used to mediate the competitive release of the ligand-dye/drug is the disaccharide, maltose. For both example platforms, the concept of maltose-induced release is demonstrated in both a microplate format as well as in living cells. After addition of maltose to the assembled donor-acceptor complex, changes in PL intensity for the donor and acceptor are monitored over time as a function of maltose concentration. An increase in donor emission correlates to the concomitant release of the ligand from the NP construct.

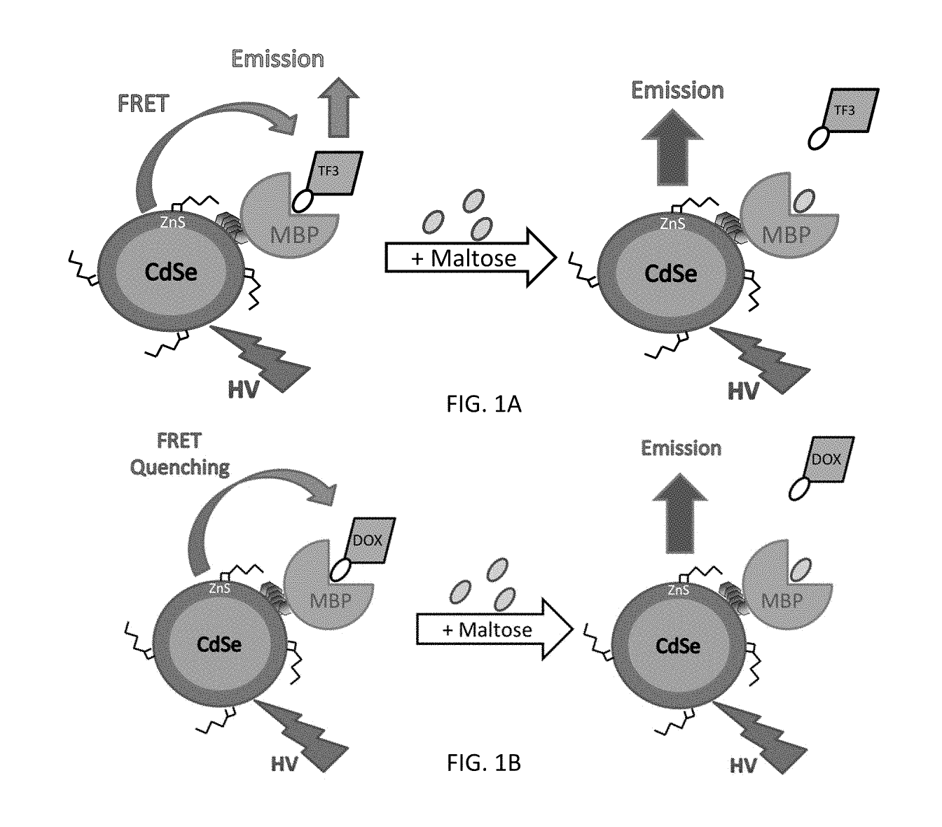

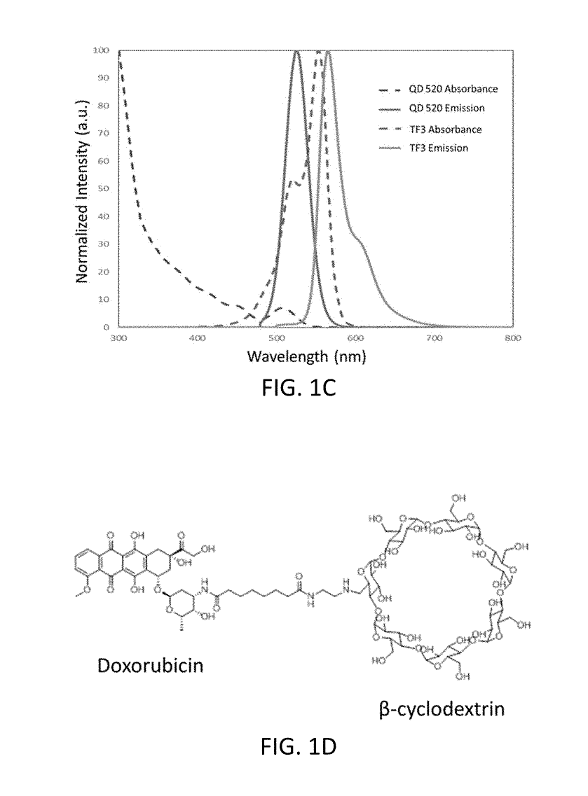

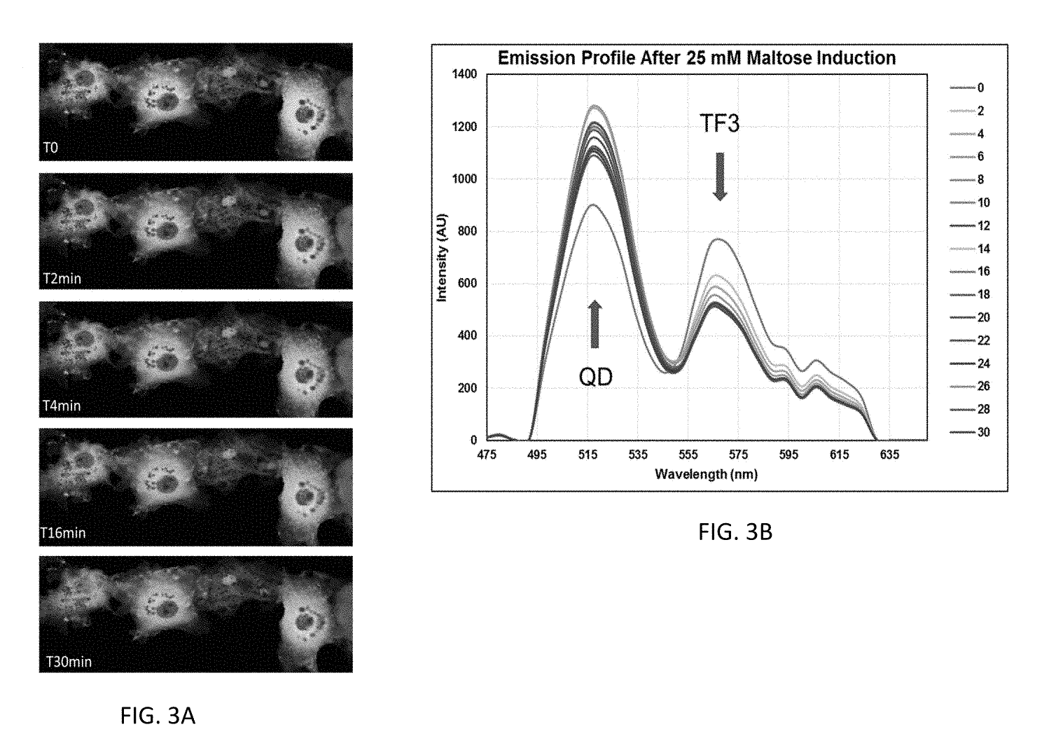

As shown in FIG. 1A, a luminescent quantum dot (QD) serves as the central scaffold onto which is assembled multiple copies of maltose-binding protein (MBP). Metal-affinity coordination driven by the polyhistidine tail on the MBP mediates self-assembly of the MBP to the QD surface. The cycloamylose, β-cyclodextrin (BCD), that is conjugated to either an emissive dye (e.g. Tide Fluor 3 (TF3) (FIG. 1A)) or a non-emissive drug (e.g., doxorubicin (DOX) (FIG. 1B)). When assembled, the QD and dye/drug moiety engage in FRET such that when the ligand-dye/drug is in the MBP binding pocket, the significant spectral overlap of the QD donor emission with the dye/drug acceptor absorbance (see FIG. 3C for QD and TF3 spectral overlap) results in quenching of the QD donor and enhanced emission of the TF3 dye acceptor. As DOX is poorly emissive, only quenching of the QD donor is observed. Upon addition of the disaccharide, maltose, to the extracellular medium, maltose enters the cells (presumably via membrane-resident α-glucoside transport systems) and acts as a competitive structural analog to drive the release of the BCD-dye/DOX ligand from the MBP binding pocket. This active actuation platform affords spatial and temporal control over cargo release from the nanoassembly by the simple addition of an innocuous, competitive ligand to the extracellular medium.

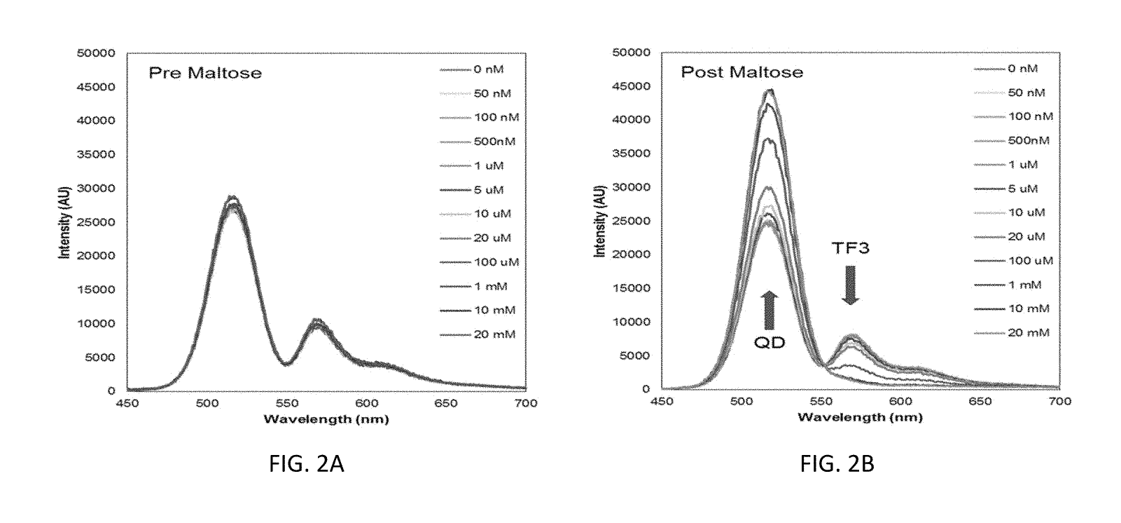

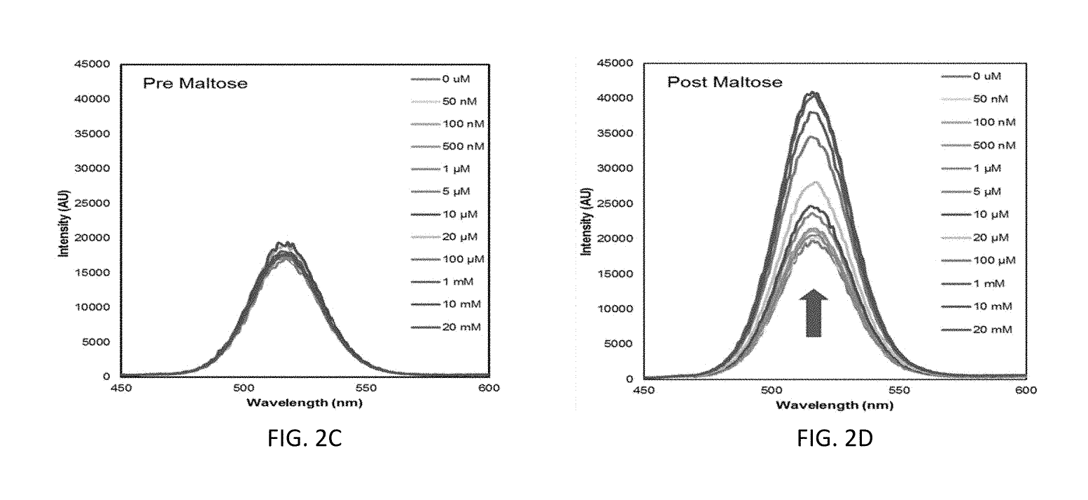

This functionality of the QD-MBP-BCD-dye/drug conjugate system was assessed using a fluorescence plate-based assay to monitor the donor/acceptor emission intensities as a function of addition of the displacing maltose ligand to solutions containing the intact complex. FIGS. 2A-2D show results of the FRET assays for both the BCD-TF3 (FIGS. 2A and 2B) and BCD-DOX (FIGS. 2C and 2D) systems. As evidenced in the spectral plots, prior to addition of increasing concentrations of maltose to the solution, strong quenching of QD PL coupled with TF3 emission. Upon addition of maltose, a concomitant ratiometric increase in QD donor re-emission and decrease in TF3 acceptor emission is observed. In the BCD-DOX system, since the DOX is poorly emissive, only re-emission from the QD donor is seen in response to addition of maltose. Similar results were obtained for the BCD-DOX ligand system.

The utility of these systems was further confirmed in the context of living cells. As shown in FIGS. 3A and 3B, the preformed ensemble QD-MBP-BCD-TF3 conjugates were microinjected into the cytosol of COS-1 cells. Maltose (25 mM) was added to the extracellular medium and cellular images were collected in spectral mode by confocal microscopy to observe the 475-640 nm spectral window as a function of time after addition of maltose. FIG. 3A shows spectral images collected at various times after maltose addition. The color progression as a function of time clearly shows the change from yellow (indicative of FRET-sensitized emission of the TF3 fluorophore) to green (indicating the re-emission of the QD donor) as increasingly more BCD-TF3 ligand-dye is displaced from the MBP binding pocket, which is driven by the internalization of the added maltose through α-glucoside transporters in the cell's plasma membrane. This is one of the key novel features of the active actuation of the NP-cargo system: the simple yet spatiotemporally controlled release of the NP-appended cargo by addition of a competitive structural analog to the extracellular environment.

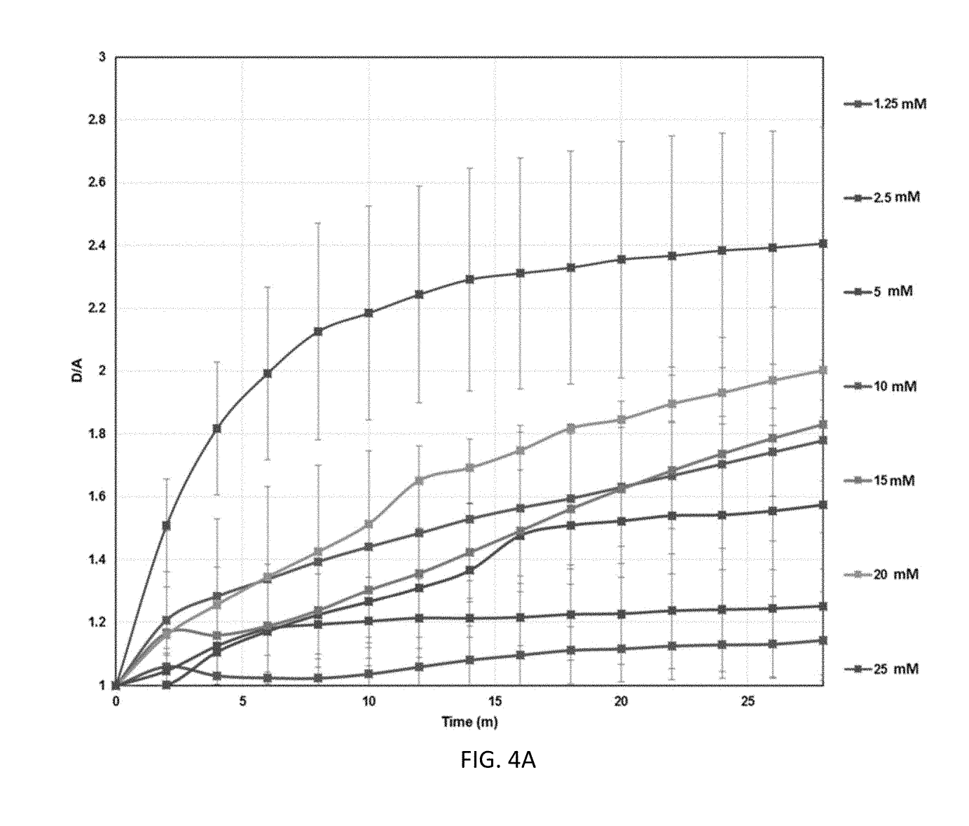

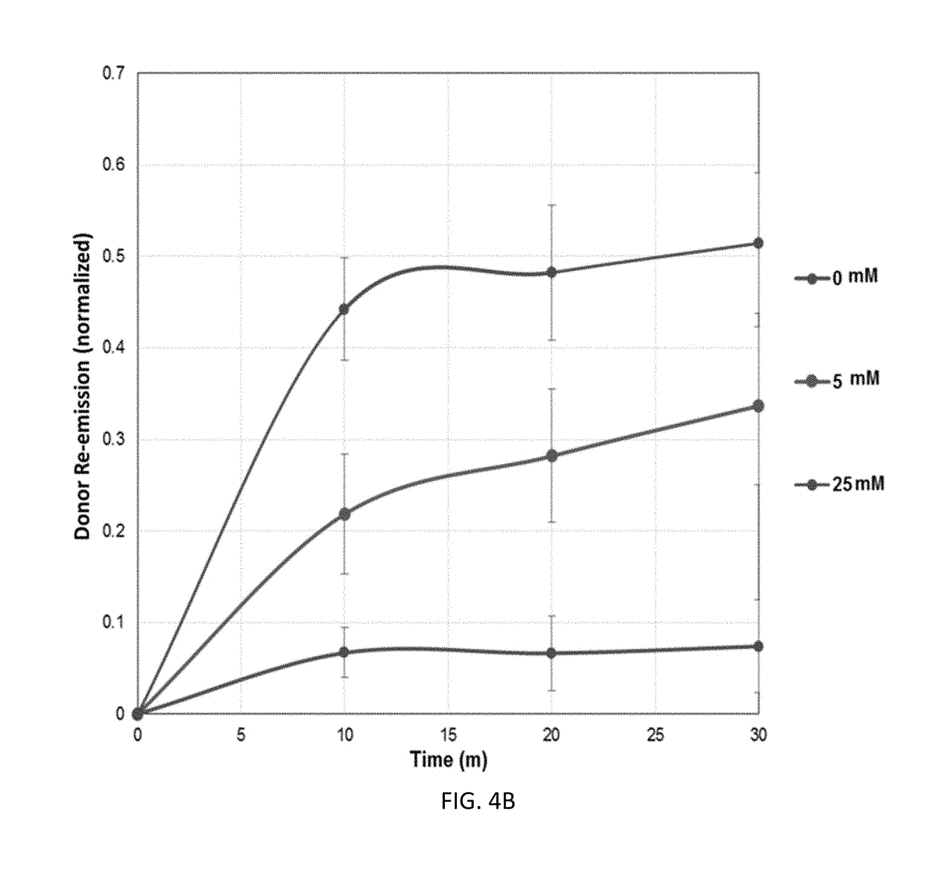

To demonstrate the quantitative nature of the maltose-induced release of the BCD-ligand systems in the intracellular environment, ratiometric analysis was performed on both systems and plotted as a function of both maltose concentration and time post-maltose addition. FIGS. 4A and 4B shows the quantified donor/acceptor ratios for the QD-MBP-BCD-TF3 and QD-MBP-BCD-DOX systems, respectively.

### Further Embodiments

The active actuation of the nanoconjugate system is demonstrated here for mammalian cells but the concept is applicable to similar delivery in other cells types prokaryotic cells and other forms of eukaryotic cells. In addition to use in vitro, this technique might be used for delivery of cargo to a living organism by providing the organism with the construct and then triggering release of the cargo.

It is expected that besides maltose binding protein, other molecules might be used as bridging structures. Conceivably the system can work with any molecule that can be made to bear (1) tag on one end to bind it to the NP (for example, polyhistidine for attachment to a metallic nanoparticle such as a QD) and (2) a binding pocket/domain to hold the cargo until induced displacement by the addition of a competitive ligand. Such molecules might include: other bacterial periplasmic binding proteins, engineered aptamers (nucleic acid or peptide), antibodies, lectins, and polymers.

The ligand that allows the cargo to releasably bind to the bridging structure (e.g., a β-cyclodextrin ligand) can be attached to the ligand in a variety of ways, preferably via a covalent bond.

In various embodiments, the second biding element can be distinct from the first binding element. For instance, in the above examples, the polyhistidine tail of MBP is distinct from the binding pocket.

### Advantages

The constructs described herein can be easily assembled, without requiring complicated synthesis or purification once the nanoassembly is made. The central NP scaffold can be further decorated with moieties (drugs, DNA, peptides) for specific cellular targeting.

Actuation of delivery can be accomplished using an innocuous, nontoxic biomolecule that is simply added to the cellular media, thus providing significant benefits over NP actuation systems using light (suffering from limited tissue depth penetration) or magnetic field (which requires expensive instrumentation to apply fields of adequate strength).

The modular design can allow for multiple substrates and associated protein carriers for more sensitive release capabilities

A competitive inhibitor added to the cellular media to drive competitive ligand release can be modified to alter its binding affinity in relation to the original substrate and therefore the release kinetics can be further tuned for various applications.

Further advantages exist with regard to the above-described QD-Maltose Binding Protein-BCD form of construct. The binding affinities of the maltose and β-cyclodextrin for the MBP binding pocket are close enough that the delivery of maltose competitively releases the BCD. The use of the QD and BCD-TF3 FRET pair allows for intracellular monitoring of ligand release in response to extracellular delivery of maltose; the large two-photon cross section of the QDs would increase the imaging capabilities of the system in deep tissue. Doxorubicin is a commonly used chemotherapy agent that has significant off-target toxicity when delivered systemically; the ability to controllably actuate the release of this drug only after the addition of maltose can reduce these issues. Additionally, doxorubicin is a quencher for the QD donor and so intracellular monitoring of release can occur before cellular morphology begins to indicate onset of cellular necrosis. The use of maltose competitive structural analog for the actuated ligand release is a nontoxic, non-invasive actuation modality that involves simple addition of maltose to cellular media. The analogous approach in vivo would be via ingestion or injection. The bridging maltose-binding protein is amenable to assembly across multiple NP scaffolds that can facilitate energy transfer for visualization.

### CONCLUDING REMARKS

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

What is claimed is:

1. A nanoparticle construct comprising:
a nanoparticle configured as a central scaffold;
a molecule configured as a bridging structure and comprising a first binding element configured for attachment to the nanoparticle and a second binding element; and
a cargo incorporating a ligand configured to allow for releasable binding of the cargo from the second binding element of the bridging structure.

2. The construct of claim 1, wherein the nanoparticle is a quantum dot.

3. The construct of claim 1, wherein the molecule configured as a bridging structure is maltose binding protein.

4. The construct of claim 1, wherein the ligand is β-cyclodextrin.

5. The construct of claim 1, wherein the cargo comprises TideFluor3 or doxorubicin.

6. A nanoparticle construct comprising:
a quantum dot configured as a central scaffold;
maltose binding protein configured as a bridging structure and comprising a domain for binding to the quantum dot and a binding pocket; and
a cargo incorporating a β-cyclodextrin ligand configured to allow for releasable binding of the cargo from the binding pocket of the maltose binding protein.

7. The nanoparticle of claim 6, wherein the cargo comprises TideFluor3 or doxorubicin.

8. A method of delivery comprising:
providing a nanoparticle construct comprising a nanoparticle configured as a central scaffold, a molecule configured as a bridging structure and comprising a first binding element configured for attachment to the nanoparticle and a second binding element, and a cargo incorporating a ligand configured to allow for releasable binding of the cargo from the second binding element of the bridging structure; and
causing the construct to be contacted with an analog of the ligand, thereby resulting in release of the cargo from the construct.

9. The method of claim 8, further comprising monitoring the release with Förster resonance energy transfer (FRET).

10. The method of claim 8, wherein the nanoparticle is a quantum dot.

11. The method of claim 8, wherein the molecule configured as a bridging structure is maltose binding protein.

12. The method of claim 8, wherein the ligand is β-cyclodextrin.

13. The method of claim 8, wherein the cargo comprises TideFluor3 or doxorubicin.
